(19) United States
(12) Patent Application Publication
(10) Pub. No.: US 2023/0277467 A1
(43) Pub. Date: Sep. 7, 2023

(54) FILM-COATED TABLET

(71) Applicants: JIANGSU WANBANG BIOPHARMACEUTICALS, Xuzhou, Jiangsu (CN); SHANGHAI SUNTECH PHARMACEUTICAL CO., LTD., Shanghai (CN)

(72) Inventors: David WONG, Xuzhou (CN); Yiran XIA, Xuzhou (CN); Bin DENG, Xuzhou (CN)

(73) Assignees: JIANGSU WANBANG BIOPHARMACEUTICALS, Xuzhou, Jiangsu (CN); SHANGHAI SUNTECH PHARMACEUTICAL CO., LTD., Shanghai (CN)

(21) Appl. No.: 18/019,033

(22) PCT Filed: Feb. 18, 2021
(86) PCT No.: PCT/CN2021/076673

§ 371 Date: Jan. 31, 2023

(30) Foreign Application Priority Data
Jul. 31, 2020 (CN) 202010765383.7
Feb. 8, 2021 (CN) 202110173260.9

(51) Int. Cl. A61K 9/28

(52) U.S. Cl. CPC A61K 9/2866; A61K 9/2813; A61K 9/2826; A61K 9/2853; A61K 9/2893

(57) ABSTRACT

A gas layer is formed in a tablet core of the film-coated tablet after being immersed in acid. A mixture containing a permeation enhancer, a gas generating agent, and drug ingredients is compressed into a tablet. Then, the tablet is coated with a semipermeable film. After the film-coated tablet is immersed in acid for several seconds or longer, gas is generated from the tablet core to form a gas layer in the tablet core. Therefore, the tablet core becomes a two-layered structure, and the tablet floats. One of the layers is the gas layer. During a research process, the tablet core generates gas and expands, causing the film to rupture. After the film is ruptured, the tablet core can continue to float, and the drug release rate is related to the composition of the tablet core and is not affected by the film.

### TECHNICAL FIELD

The present disclosure relates to a film-coated tablet, which belongs to the field of preparations.

### BACKGROUND

Since gastric floating preparations require rapid water penetration to produce gas rapidly, most effervescent floating systems do not employ film coating techniques, but such effervescent floating systems often suffer from insufficient suspension force due to gas leakage. Therefore, some documents have proposed the film coating floating techniques.

U.S. Pat. Nos. 8,580,303 and 8,333,991 have reported a dosage form, which includes (a) at least one composition including a gas generating agent and gabapentin, and (b) at least one hydrophilic film coated with (a). The hydrophilic film expands by expansion and floats on the gastric juice, and the gastric juice can permeate the hydrophilic film. U.S. Pat. Nos. 8,529,955, 8,440,232 and 8,475,813 have proposed a dosage form, which includes gabapentin and a pharmaceutically acceptable excipient-containing tablet core, and a semipermeable film surrounding the tablet core, the semipermeable film includes a plasticiser, and is permeable to gastric juice but substantially impermeable to insoluble gabapentin. Nilesh Desai et al. (AAPS PharmSciTech 2017 October; 18(7): 2626-2638) and Jin Guan et al. (Int. J. Pharm. 383(2010) 30-36) also introduced the use of penetrating tablets in gastric retenting dosage forms. However, since drug release needs to pass through porous channels of the coating film, the drug must be dissolved in a medium in a molecular state. Insoluble drug particles cannot be released through the film, and the technology may have the problem of low release rate when applied to such drugs.

Sheng-Feng Hung et al. (PLoS One. 2014:9(6): e100321) applied a polymer coating containing a plasticizer to a multi-unit floating drug delivery system. Vinay Kumar Katakam et al. (Trop. J. Pharm. Res. April 2014; 13(4): 489-496) taught a technique for applying a three-layer coating to the tablet surface, including an isolating layer, an effervescent layer (a gas generating layer that does not generate gas inside the tablet core), and an outer polymer membrane layer. However, coating the outside of the tablet core may delay the tablet floating starting time. At the same time, gas generated at the periphery of the tablet core will hinder drug release and further cause inter-batch dissolution fluctuations.

Ampanart Huanbuta et al. (PharmSciTech, Volume 17, Issue 3, June 2016) introduced a novel floating system with a tablet core being surrounded by a semipermeable film. The tablet core contained a high proportion of water-insoluble microcrystalline cellulose or a gas generating agent. During the dissolution process, the generated gas was uniformly distributed in the tablet core (refer to FIG. 4 and FIG. 8 of the product). This uniform distribution of the gas within the tablet core or around the film coating will hinder drug release and cause fluctuations in drug release.

Zulfequar A. Khan et al. (AAPS Pharmsitech, Volume 12, Issue 4, December 2011) introduced a floating tablet that coats a drug-loaded tablet core with a gas generating layer and a polymer layer. In this way, a polymer membrane can act as a barrier to prevent the instantaneous penetration of gastric juice into the membrane, thus delaying the conversion of sodium bicarbonate into carbon dioxide. The drug is released through drug releasing holes. Rania A. H. Ishak (J Pharm Pharm Sci, 18(1)77-100, 2015) suggested that preferred drug candidates for gastric retenting preparations are those that are easily soluble in the gastric acid environment. In the same view as Khan, Raina also considered that the polymer coating should be able to withstand the pressure generated by carbon dioxide to avoid rupture. Sadhana Shahi et al. (Asian Journal of Pharmaceutical Technology & Innovation, 03(15); 2015; 32-49) believed that a drug needs to be released from drug releasing holes in a polymer membrane of a tablet, and the size of the drug releasing hole was from 600 microns to 1 millimeter. They also believed that for basic osmotic pump tablets (single-layer osmotic pump tablets), water-soluble drugs were preferred. These documents all agree that the coating needs to remain intact and unruptured, because once it is ruptured, it will lead to gas leakage and suspension force problems; however, because the size of the drug releasing hole is limited, in order to achieve the desired drug release effect, this type of dosage form is usually suitable for drugs with good water solubility or gastric acid solubility, but is not suitable for insoluble drugs.

### SUMMARY

In experiments, the inventors have found that film rupture is an effective method to release insoluble drugs from a drug layer of a floating tablet, and it is surprisingly found that for a film-coated tablet developed based on the method accordingly, the tablet can continue to float after the film is ruptured, and will not disintegrate to release the drug.

In our study, we have found that when a gas generating tablet core prescription is not coated, immersion in 0.1N HCl may result in rapid gas release and disintegration, and stable flotation and sustained release cannot be realized. Then the film is coated with a special material, although the gas can be wrapped and floats quickly, it is difficult to release acid-insoluble drugs even if there are drug releasing holes due to the blocking effect of a coating film. In one study, we unexpectedly find that after the film is ruptured, the tablet core can not only continue to float, but also the drug release rate is only related to the composition of the tablet core and is not affected by the film. According to this study, the present disclosure provides a film-coated tablet. The film-coated tablet includes a tablet core and a semipermeable film. The tablet core includes the following ingredients in percentage by weight: 2% to 15% of a drug, 3% to 50% of a penetration enhancer, 10% to 50% of a gas generating agent, 10% to 45% of a water-soluble swellable polymer and an excipient, and a sum of the ingredients is 100%; the semipermeable film includes 20% to 80% of a water-insoluble polymer, 10% to 85% of micronized water-soluble particles and 0% to 40% of other additives, and a sum of the ingredients is 100%; and a coating weight gain of the semipermeable film is not more than 5%.

The gas generating agent and penetration enhancer described herein are uniformly distributed in the tablet core, and in 0.1N HCl, the tablet expends and floats on the surface. Because the viscosity of the tablet core described herein is not particularly high after absorbing water, a gas layer is gathered and formed at one side of the tablet core, the tablet core expands continuously, and finally the semipermeable film is ruptured. It is surprising that after the semipermeable film is ruptured, the tablet continues to float, and the drug is released along with the viscosity of a tablet core skeleton, and no core disintegration dose dumping occurs.

In some examples of the present disclosure, the crack of the semipermeable film occupies at least 10% of the surface of the tablet.

According to the film-coated tablet of the present disclosure, the semipermeable film formed by the compositions is a water-permeable semipermeable film, and in some preferred examples, the coating weight gain of the semipermeable film is greater than or equal to 2% but less than or equal to 5%, for example, the coating weight gain of the film is greater than or equal to 2% but less than 5%. The inventors have found that when it is higher than this range, it will be more difficult to rupture the semipermeable film, and when it is lower than this level, a phenomenon of uneven coating is likely to occur during the pharmaceutical process.

After the film-coated tablet of the present disclosure is immersed in an acidic medium, the water-permeable semipermeable film can enable the tablet core to quickly form a gas layer and quickly float within seconds to minutes, thereby effectively avoiding the risk of entering the small intestine, and the tablet core continues to expand subsequently to rupture the semipermeable film, but the tablet core can continue to float for 6 hours or longer, thereby realizing the sustained release of drugs.

In some specific examples, the tablet core includes the following ingredients in percentage by weight: 2% to 15% of the drug, 3% to 25% of the penetration enhancer, 10% to 25% of the gas producing agent, 10% to 45% of the water-soluble swellable polymer, 4% to 6% of a binder, 0.2% to 5% of a solubilizer, 1% to 50% of a filler, 0.5% to 1.5% of a glidant, 0.5% to 1.5% of a lubricant and 0% to 5% of a disintegrant, and a sum of the ingredients is 100%.

The drug of the present disclosure can be applied to any drug, and in particular, it can be applied to a drug with low solubility in acid, and the drug with low solubility in acid in the present disclosure refers to drugs with solubility of less than 1 mg/ml in hydrochloric acid with pH of 1.2. The dosage form of the present disclosure can meet mass sustained release of the drug after the film is ruptured, which can not only meet the release requirements, but also avoid excessive release of disintegration. In some examples, drugs with low solubility in acid include, but are not limited to, cabozantinib or salt thereof, lenvatinib or salt thereof, or crizotinib or salt thereof; and in some examples, a preferred weight ratio is 2% to 15%.

The drug of the present disclosure may be ground or dispersed in a substance prior to forming the dosage form, so as to improve the dissolution rate. In some examples, the drug form may be ground to a micron or nanometer scale, for example, the average particle size may be from 0.1 microns to 20 microns. In some other examples, solid dispersion, clathrate, and other techniques may be adopted to improve the dissolution rate. These means may be common means in the art, for example Zhi Hui Loh et al. (Asian Sciences, Volume 10, Issue 4, July 2015, 255-274.) described different grinding techniques for improving the dissolution rate of water-insoluble drugs. Vincent Caron et al. (Mixer Mill MM400, Retsch GmbH & Co., Germany) performed milling at room temperature with a PM 100 high-energy planetary mill (Retsch, Germany) and performed frozen grinding in an oscillating ball mill. Methods of molecular dispersion (here, it is equivalent to solid dispersants) were also described in a number of scientific articles (e.g. AAPS PharmSci Tech. 2013 March; 14(1): 464-474.). Francimary L. Guedes et al. (AAPS PharmSci Tech. 2011 March; 12(1): 401-410.) used polyvinylpyrrolidone (PVP) and polyethylene glycol (PEG) to prepare solid dispersants for novel drugs. Drug and polymer were dissolved in a methanol/chloroform mixture and then a solvent was removed by evaporation and freeze drying. The drug was in an amorphous state. Lili Fitriani et al. (J Adv. Pharm Technol.). Res.2016 July-September; 7(3): 105-109), Efavirenz-PVP K30 dispersants were prepared by a solvent evaporation method at ratios of 2:1, 1:1, and 1:2 and dried using a freeze dryer. Abhishek Singh et al. (Advanced Drug Delivery Review, Volume 100, May 1, 2016, 27-50) prepared an amorphous solid dispersant by spray drying.

In some examples of the present disclosure, the penetration enhancer includes, but is not limited to, a water-soluble salt of an inorganic acid, such as one or more selected from potassium chloride, potassium sulfate, potassium hydrogen phosphate, sodium hydrogen phosphate, sodium chloride, citric acid or tartaric acid; in some specific examples, the penetration enhancer is one or more selected from sodium chloride, potassium chloride or citric acid; and in some specific examples, a weight ratio of the penetration enhancer is 3% to 25%.

In some examples of the present disclosure, the gas generating agent is sodium bicarbonate; and in some specific examples, a weight ratio of the gas generating agent is 10% to 50%.

In some examples of the present disclosure, the water-soluble swellable polymer is selected from hydroxypropyl methylcellulose, carbomer or polyethylene oxide, methylcellulose, gelatin and other water-soluble polymers with similar high viscosity; and in some specific examples, a weight ratio of the water-soluble swellable polymer is 10% to 45%.

The binder of the present disclosure includes, but is not limited to, starch such as potato starch, wheat starch, corn starch; natural gum such as Arabic gum, alginic acid, guar gum; liquid glucose, povidone, polyethylene oxide, polyvinylpyrrolidone, poly-N-vinylamide, polyethylene glycol, gelatin, polypropylene glycol, tragacanth, a combination thereof, and other materials and mixtures thereof known to those of ordinary skill in the art. In some examples, the binder is added in an amount of 4% to 6%.

The solubilizer of the present disclosure includes, but is not limited to, a surfactant such as polysorbate 80 (sold under the trade name TWEEN® 80) and the like, a complexing agent such as β-cyclodextrin and the like, a polymer such as poloxamer 188 and the like and a co-solvent such as methanol and the like. The solubilizer may also be an acid or a base if the solubility of the drug is pH dependent. In some examples, the solubilizer is added in an amount of 0.2% to 1%.

The filler of the present disclosure includes, but is not limited to, mannitol, fructose, sucrose, lactose, xylitol, sorbitol, microcrystalline cellulose, calcium carbonate, calcium hydrogen phosphate or ternary calcium, calcium sulfate, and the like. In some examples, the filler is added in an amount of 1% to 50%.

The glidant of the present disclosure includes, but is not limited to, silica, magnesium trisilicate, tribasic calcium phosphate, calcium silicate, magnesium silicate and other materials known to those of ordinary skill in the art. In some examples, the glidant is added in an amount of 0.5% to 1.5%.

The lubricant of the present disclosure may be selected from, but not limited to, those conventionally known in the art, such as sodium stearyl fumarate, magnesium stearate, aluminum stearate or calcium stearate or zinc stearate, polyethylene glycol, glyceryl monostearate, glyceryl monostearate, glyceryl behenate, mineral oil, sodium stearyl fumarate, stearic acid, hydrogenated vegetable oil and talc. In some examples, the lubricant is added in an amount of 0.5% to 1.5%.

The disintegrant of the present disclosure may be selected from, but not limited to, those conventionally known in the art, such as croscarmellose sodium. The disintegrant may or may not be added.

In some examples of the present disclosure, the semipermeable film includes 20% to 80% of the water-insoluble polymer, 10% to 85% of the micronized water-soluble particles, and 0% to 40% of other additives, and a sum of the ingredients is 100%.

In some examples, the water-insoluble polymer includes, but is not limited to, one or more of polyvinyl alcohol, polyurethane, ethyl cellulose, methyl cellulose, cellulose acetate, cellulose acetate butyrate, polyvinyl chloride, polycarbonate, ethylene-vinyl acetate, polymethyl methacrylate, or polypropylene. In some specific examples, the water-insoluble polymer is cationic polymethyl methacrylate (commercial products such as EUDRAGIT RL® and EUDRAGIT RS®), and a preferred dosage range is 30% to 70%.

In some examples, the micronized water soluble particles are water soluble molecules or water soluble salts or mixtures thereof in a solid particulate state during the preparation of a coating mixture, for example including but not limited to one or more of sodium chloride, sodium bicarbonate or mannitol; a preferred micronized water soluble particle is mannitol, which is present in the form of microparticles during the preparation of the coating mixture. In some examples, the microparticles have an average particle size of 0.1 microns to 20 microns; and a preferred dosage range is 10% to 70%.

In a specific example, other additives of the present disclosure may include plasticizer or/and anti-sticking agent, the plasticizer includes, but is not limited to, dibutyl phthalate, triethyl citrate, polyethylene glycol (PEG) and the like; the anti-sticking agent includes, but is not limited to, talc, stearic acid, magnesium stearate, or colloidal silica or similar materials; and a preferred dosage range is 0% to 30%.

The film of the present disclosure may also include a colorant, for example including, but not limited to, pharmaceutical grade dyes and pigments, red iron oxide, yellow iron oxide, titanium dioxide, carbon black, and indigo. In some examples, the colorant is added in an amount of 0% to 0.5%.

The final pharmaceutical dosage form of the present disclosure may also optionally have one or more additional coatings (in addition to the semipermeable coating), such as a moisture-proof film coating, a sugar coating, and other coatings known in the art. The coating is not considered to be a matrix in the present disclosure. Coating materials that can be used in a coating process (for the finished dosage form) include, but are not limited to, polysaccharide such as maltodextrin, alkylcellulose such as methylcellulose or ethylcellulose, cellulose acetate, hydroxyalkylcelluloses (e.g., hydroxypropylcellulose or hydroxypropylmethylcellulose); polyvinylpyrrolidone, Arabic gum, corn, sucrose, gelatin, shellac, cellulose acetate, phthalate, lipid, synthetic resin, acrylic polymer, an OPADRY® coating system, polyvinyl alcohol (PVA), a copolymer of vinyl pyrrolidone and vinyl acetate (for example sold under the trade name PLASDONE®) and polymer based on methacrylic acid (such as those sold under the trade name EUDRAGIT®). These may be applied from aqueous or non-aqueous systems or a combination of aqueous and non-aqueous systems (as appropriate). Additives may be included with a film-forming agent to obtain a satisfactory film. These additives may include a plasticizer such as dibutyl phthalate, triethyl citrate, polyethylene glycol (PEG) and the like, a channel forming agent such as surfactant, short-chain water-soluble polymer, salt and the like, an anti-sticking agent such as talc, stearic acid, magnesium stearate, and colloidal silica and the like, and a filler such as talc, precipitated calcium carbonate, a polishing agent such as beeswax, carnauba wax, synthetic chlorinated wax, and an opacifying agent such as titanium dioxide, and the like. All these excipients may be used at levels well known to those skilled in the art.

The present disclosure also discloses a method for preparing the film-coated tablet of the present disclosure. The method includes the following steps of: mixing tablet core materials, tabletting, and coating to form a semipermeable film.

The specific operations involved in the preparation method of the present disclosure may be performed according to conventional methods in the art. For example, the tablet core of the present disclosure may be formed by direct compression, pelletization-compression, pellet-compression or equivalent methods. In direct tabletting, the raw materials are fully mixed and placed in a compression mold and compressed to form tablets. During pelletization, a formulation solution is sprayed onto a mixture of “particles” and excipients to form particles. The particles are dried and ground to a required particle size distribution. The particles are then mixed with other excipients and placed in a compression mold and compressed to form tablets. A technique for preparing tablets is described in Remington's Pharmaceutical Sciences, (Arthur Osol, editor), 1553-1593 (1980). Particle coating using a fluidized bed is reported in U.S. Pat. No. 8,282,957, which described particle spray using a spray drying process. U.S. Pat. No. 8,911,766 described particle spraying using a solvent evaporation technique. Some other alternative methods may also be used for the particle or microparticle coating in the present disclosure. Tablet cores are coated with a coating pan, a fluidized bed, or similar equivalent equipment. Disc coating may be a convenient method for film coating of the tablet core. The tablet cores are placed in a disc, the disc is rotated, and a semipermeable polymer solution is sprayed onto the tablet cores. Other spraying techniques, such as an air suspension program (fluidized bed), may also be considered. The tablet cores are suspended in air in a fluidized bed and virtually sprayed with a polymer solution through the circulation of a Wurster column. Pan coating programs can be found in the United States. Patent application No. 20060099262. Air suspension program is described in U.S. Pat. No. 2,799,241. Am. Pharm. Assoc., Volume 48, pp. 451-459 (1959); and ibid., Volume 49, pp. 82-84 (1960).

The present disclosure has the beneficial effects that:

The pharmaceutical dosage form of the present disclosure is the film-coated tablet, which includes the gas generating tablet core and the semipermeable film; when the dosage form is immersed in acid, the tablet core generates gas, and the tablet core becomes a two-layered structure and rapidly forms a floating state. The tablet core continues to generate gas and expands, causing the semipermeable film to rupture, a tablet core skeleton is exposed and continues to keep the floating state, the drug is released along with the characteristics of a skeleton material, and compared with the drug releasing hole, drug release requirement of the insoluble drug can be met, and the disintegration dose dumping of the tablet core due to film rupture are avoided. In the experiment, we find that film rupture is an effective method for the release of insoluble drugs from gastric floating film-coated tablets. In the experiment, it is surprising that the tablets continue to float after the film is ruptured and no dose dumping occurs. Drug release is related to the composition of the tablet core.

Unless otherwise specified, the terms of the present disclosure is defined as follows:

“Optional” or “optionally” means that the subsequently described circumstance may or may not occur, such that the description includes instances in which that circumstance occurs as well as instances in which that circumstance does not occur.

The singular form (such as “a”, “an” and “the”) included in claims include plural references unless specifically stated or the context clearly dictates otherwise. On the other hand, the singular form “ONE” does not include plural references.

“Consisting of” is a transitional phrase used in claims of the present disclosure. “Consisting of” excludes any element, step, or ingredient unspecified in claims.

“Gas layer” means gas sites are formed within a coated tablet when the coated tablet is immersed in an acidic medium. The gas cannot be uniformly distributed within the tablet core.

The term “carbonate” may be exchanged for “bicarbonate”, which may be carbonate or bicarbonate.

The term “semipermeable film” may be exchanged for “film” or “semipermeable coating”, unless it is specifically intended for a decorative coating.

“Film crack” means cracks formed by gas formation in the tablet core or/and pressure generated by tablet core expansion when the film-coated tablet is immersed in 0.1N HCl. The size is greater than 2 millimeters or at least 10% of the surface of the tablet.

### BRIEF DESCRIPTION OF THE DRAWINGS

FIG. 1 shows that a coated tablet is immersed in 0.1N HCl and begins to float within 3 minutes, and gas generated by the tablet core finally forms a gas layer.

FIG. 2 shows that a coated tablet expends to rupture a film; A is a side view with at least 10% of a surface of the tablet being exposed from the film crack; and B is a top view with at least 10% of the surface of the tablet being exposed from the film crack.

FIG. 3 shows that the tablet core further expands, and most of the tablet core “leaks” out of a film.

### DETAILED DESCRIPTION

The foregoing examples are illustrative examples of the present disclosure and are exemplary only. Variations and modifications may be made by those skilled in the art without departing from the spirit and scope of the present disclosure. All such modifications and variations are intended to be included within the scope of the present disclosure.

The experimental methods in the following examples are all conventional methods unless otherwise specified. The test materials used in the following examples are purchased from conventional biochemical reagent stores unless otherwise specified.

### Example 1

Two batches were prepared. The prescription is as follows:

| | 210112-FC1 | 210112-FC2 |
|---|---|---|
| Tablet core | 5 | 6 |
| Cabozantinib malate | 10 | 10 |
| Povidone K30 | 4.67 | 4.67 |
| Tween-80 | 0.5 | 0.5 |
| Hydroxypropyl methylcellulose | 30 | 45 |
| K100M | | |
| Lactose | 16.83 | 1.83 |
| Sodium chloride | 13 | 13 |
| Sodium bicarbonate | 20 | 20 |
| Citric acid | 3 | 3 |
| Silicon dioxide | 1 | 1 |
| Sodium stearyl fumarate | 1 | 1 |
| % | 100 | 100 |
| Tablet weight mg | 600 | 600 |
| Coating | | |
| EUDRAGIT RL PO | 65.6% | 65.6% |
| Triethyl citrate | 9.8% | 9.8% |
| Mannitol | 16.4% | 16.4% |
| Talc | 8.2% | 8.2% |
| Solid content | 9.93% | 9.93% |
| Coating weight gain | 2.50% | 2.50% |

The preparation process is as follows:

Mannitol jet milling: mannitol was taken for jet milling, and the particle size was D(90)=15.3 μm.

Tablet core: other tablet core materials except sodium stearyl fumarate were weighted according to a prescription amount, sieved with a 40-mesh sieve, and uniformly mixed in a post-placed mixer, sodium stearyl fumarate with a prescription amount was added, and the materials were uniformly mixed. The evenly mixed tablet was compressed by a rotary tablet press, stamped using a 11.5 mm round front concave punch with a main pressure of 7-10 KN, and tabletted.

Coating: a prescription amount of isopropanol and water were taken, and mixed evenly, ¾ of the solvent was taken and added with a prescription amount of Eudragit RL PO, and stirred for dissolving (NLT60 min) to form a clear solution; the other ¼ of the solvent was taken and added with a prescription amount of TEC, a prescription amount of mannitol and a prescription amount of talc, dispersed evenly and then added into the Eudragit solution, and the materials were stirred and mixed evenly. The prepared tablet core was coated by a high-efficiency coating pan, and the coating weight gain was 2.5%.

Result:

The tablets were placed in 0.1N HCl at 37° C. 210112-FC1 started to float within 5 seconds and the coating started to rupture within 30 seconds. While 210112-FC2 started to float within 1 minute and the coating started to rupture within 1 minute.

### Example 2

Six batches were prepared. The preparation process was the same as that in Example 1, the coating prescription was the same, and the coating weight gain was 3.8% to 4.6%.

| | 210115- | 210115- | 210115- | 210105- | 210105- | 210105- |
|---|---|---|---|---|---|---|
| | FC1 | FC2 | FC3 | FC1 | FC2 | FC3 |
| Cabozantinib malate | 10 | 10 | 10 | 10 | 10 | 10 |
| Povidone K30 | 4.67 | 4.67 | 4.67 | 4.67 | 4.67 | 4.67 |
| Tween-80 | 0.5 | 0.5 | 0.5 | 0.5 | 0.5 | 0.5 |
| Hydroxypropyl | 45 | 45 | 45 | 15 | 20 | 10 |
| methylcellulose | | | | | | |
| K100M | | | | | | |
| Lactose | 11.83 | 6.83 | 14.83 | 31.83 | 14.83 | 46.83 |
| Sodium chloride | 13 | 13 | 0 | 13 | 20 | 7 |
| Sodium bicarbonate | 10 | 15 | 20 | 20 | 25 | 16 |
| Citric acid | 3 | 3 | 3 | 3 | 3 | 3 |
| Silicon dioxide | 1 | 1 | 1 | 1 | 1 | 1 |
| Sodium stearyl | 1 | 1 | 1 | 1 | 1 | 1 |
| fumarate | | | | | | |
| % | 100 | 100 | 100 | 100 | 100 | 100 |
| Tablet weight mg | 600 | 600 | 600 | 600 | 600 | 600 |
| Coating (%) | | | | | | |
| EUDRAGIT RL PO | 66 | 66 | 66 | 66 | 66 | 66 |
| Triethyl citrate | 10 | 10 | 10 | 10 | 10 | 10 |
| Mannitol | 16 | 16 | 16 | 16 | 16 | 16 |
| Talc | 8 | 8 | 8 | 8 | 8 | 8 |
| Solid content | 11.88 | 11.88 | 11.88 | 11.88 | 11.88 | 11.88 |
| Coating weight gain | 3.86% | 3.86% | 3.86% | 4.53% | 4.53% | 4.53% |

Result:

The tablets were placed in 0.1N HCl at 37° C. The observations are as follows:

| | 210115- | 210115- | 210115- | 210105- | 210105- | 210105- |
|---|---|---|---|---|---|---|
| | FC1 | FC2 | FC3 | FC1 | FC2 | FC3 |
| Floating | 3.75 | 3.5 | 4 | 2.5 | 2.25 | 2.75 |
| started | | | | | | |
| within ( ) | | | | | | |
| minutes | | | | | | |
| Coating | 10 | 8 | 30 | 30 | 30 | 30 |
| started to | | | | | | |
| rupture | | | | | | |
| within ( ) | | | | | | |
| minutes | | | | | | |

Dissolving experiment (900 mL, 0.1 NHCl+1.0% Tween-80) paddle method (CJL-3 type sediment basket was added) 50 rpm

| Time | 210115- | 210115- | 210115- | 210105- | 210105- | 210105- |
|---|---|---|---|---|---|---|
| (h) | FC1 | FC2 | FC3 | FC1 | FC2 | FC3 |
| 0.5 | 0 | 1 | 0 | 33 | 4 | 34 |
| 1 | 0 | 1 | 0 | 70 | 8 | 68 |
| 1.5 | 1 | 1 | 0 | 78 | 10 | 74 |
| 2 | 2 | 2 | 1 | 83 | 12 | 79 |
| 3 | 3 | 3 | 2 | 88 | 15 | 86 |
| 4 | 4 | 4 | 3 | 92 | 18 | 89 |
| 6 | 6 | 7 | 5 | 97 | 24 | 95 |

### Example 3

Two batches were prepared. The preparation process was the same as that in Example 1, the prescriptions of the tablet core and coating were slightly adjusted, and the coating weight gain was about 4%.

| | 210128-FC1 | 210128-FC2 |
|---|---|---|
| Cabozantinib malate | 13.33 | 13.33 |
| Povidone K30 | 4.67 | 4.67 |
| Tween-80 | 1 | 1 |
| Hydroxypropyl methylcellulose K100M | 30 | 30 |
| Lactose | 13 | 8 |
| Croscarmellose sodium | 0 | 5 |
| Sodium chloride | 13 | 13 |
| Sodium bicarbonate | 20 | 20 |
| Citric acid | 3 | 3 |
| Silicon dioxide | 1 | 1 |
| Sodium stearyl fumarate | 1 | 1 |
| % | 100 | 100 |
| Tablet weight mg | 450 | 450 |
| Coating (%) | | |
| EUDRAGIT RL PO | 31.7 | 66 |
| Triethyl citrate | 16.7 | 10 |
| Mannitol | 51.7 | 16 |
| Solid content | 13 | 13 |
| Coating weight gain | 4% | 4% |

Result:

The tablets were placed in 0.1N HCl at 37° C. The observations are as follows:

| | 210128-FC1 | 210128-FC2 |
|---|---|---|
| Floating started within ( ) minutes | 0.9 | 0.9 |
| Coating started to rupture within ( ) minutes | 60 | 60 |

### Example 4

Two batches were prepared. The preparation process and the coating prescription were the same as those in Example 1, and the coating weight gain was 3.8% to 4.6%.

| | 2101118-FC1 | 2101118-FC2 |
|---|---|---|
| lenvatinib mesylate | 3.33 | 3.33 |
| Povidone K30 | 4.67 | 4.67 |
| Tween-80 | 0.5 | 0.5 |
| Hydroxypropyl | 35 | 45 |
| methylcellulose K100M | | |
| Lactose | 18.5 | 8.5 |
| Sodium chloride | 13 | 13 |
| Sodium bicarbonate | 20 | 20 |
| Citric acid | 3 | 3 |
| Silicon dioxide | 1 | 1 |
| Sodium stearyl fumarate | 1 | 1 |
| % | 100 | 100 |
| Tablet weight mg | 300 | 300 |
| EUDRAGIT RL PO | 65.6% | 65.6% |
| Triethyl citrate | 9.8% | 9.8% |
| Mannitol | 16.4% | 16.4% |
| talc | 8.2% | 8.2% |
| Solid content | 11.88% | 11.88% |

Result:

The tablets were placed in 0.1N HCl at 37° C. The observations are as follows:

| | 2101118-FC1 | 2101118-FC2 |
|---|---|---|
| Floating started within ( ) minutes (min) | 2.5 | 2.8 |
| Coating started to rupture within ( ) | 30 | 30 |
| minutes (min) | | |

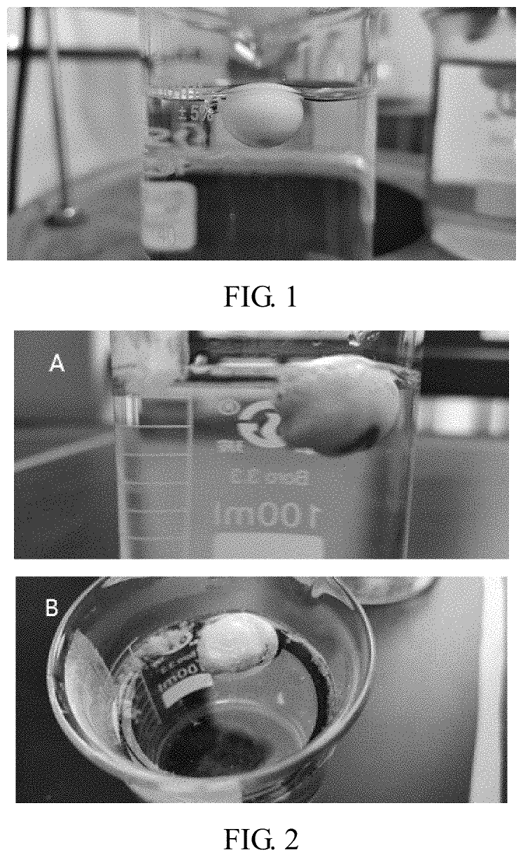

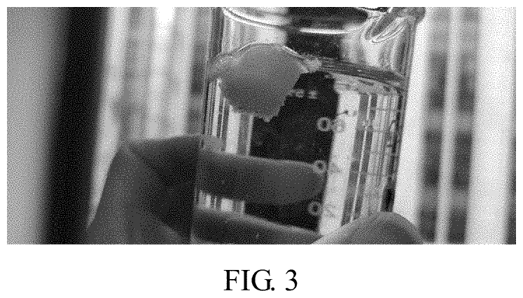

A sample (Batch No. 2101118-FC2) prepared in Example 4 floated in 0.1N HCl at 37° C. within 3 minutes, as shown in FIG. 1, and the tablet core generated gas to form a gas layer. The tablet core continued to expand, causing the film to rupture, and at least 10% of the surface of the tablet was exposed from the film crack (as shown in FIG. 2). After the film was ruptured, the tablet core could continue to float without dose dumping, and the drug was released along with the viscosity of a tablet core skeleton and was not affected by the film. After a longer period of time, the tablet core further expanded and most of the tablet core “leaked” out of the film (as shown in FIG. 3), but could continue to float for 6 hours or longer.

1. A film-coated tablet, wherein the film-coated tablet comprises a tablet core and a semipermeable film, wherein the tablet core comprises the following ingredients in percentage by weight: 2% to 15% of a drug, 3% to 50% of a penetration enhancer, 10% to 50% of a gas producing agent, 10% to 45% of a water-soluble swellable polymer and an excipient, and a sum of the ingredients is 100%; the semipermeable film comprises 20% to 80% of a water-insoluble polymer, 10% to 85% of micronized water-soluble particles, and 0% to 40% of other additives, and a sum of the ingredients is 100%; and a coating weight gain of the semipermeable film is not more than 5%.

2. The film-coated tablet according to claim 1, wherein the coating weight gain of the semipermeable film is greater than or equal to 2% but less then or equal to 5%.

3. The film-coated tablet according to claim 1, wherein the excipient is selected from one or more of a binder, a solubilizer, a filler, a glidant, or a lubricant.

4. The film-coated tablet according to claim 1, wherein the tablet core comprises the following ingredients in percentage by weight: 2% to 15% of the drug, 3% to 25% of the penetration enhancer, 10% to 25% of the gas generating agent, 10% to 45% of the water-soluble swellable polymer, 4% to 6% of the binder, 0.2% to 5% of the solubilizer, 1% to 50% of the filler, 0.5% to 1.5% of the glidant, 0.5% to 1.5% of the lubricant and 0% to 5% of a disintegrant, and a sum of the ingredients is 100%.

5. The film-coated tablet according to claim 1, wherein the drug is a drug with low solubility in acid; and the penetration enhancer is selected from one or more of potassium chloride, potassium sulfate, potassium hydrogen phosphate, sodium hydrogen phosphate, sodium chloride, citric acid or tartaric acid.

6. The film-coated tablet according to claim 1, wherein the gas generating agent is sodium bicarbonate, and the water-soluble swellable polymer is selected from hydroxypropyl methylcellulose, carbomer or polyethylene oxide.

7. The film-coated tablet according to claim 4, wherein the binder is selected from potato starch, wheat starch, corn starch, Arabic gum, alginic acid, guar gum; liquid glucose, povidone, polyethylene oxide, polyvinylpyrrolidone, poly-N-vinylamide, polyethylene glycol, gelatin, polypropylene glycol or tragacanth; the solubilizer is selected from polysorbate 80, β-cyclodextrin, poloxamer 188 or methanol; the filler is selected from mannitol, fructose, sucrose, lactose, xylitol, sorbitol, microcrystalline cellulose, calcium carbonate, calcium hydrogen phosphate or ternary calcium or calcium sulfate; the glidant is selected from silicon dioxide, magnesium trisilicate, tribasic calcium phosphate, calcium silicate or magnesium silicate; the lubricant is selected from sodium stearyl fumarate, magnesium stearate, aluminum stearate, calcium stearate, zinc stearate, polyethylene glycol, glyceryl monostearate, glyceryl monostearate, glyceryl behenate, mineral oil, sodium stearyl fumarate, stearic acid, hydrogenated vegetable oil or talc.

8. The film-coated tablet according to claim 1, wherein the semipermeable film comprises 20%-80% of the water-insoluble polymer, 10%-85% of the micronized water-soluble particles and 0%-40% of other additives, and a sum of the ingredients is 100%.

9. The film-coated tablet according to claim 8, wherein the water-insoluble polymer is one or more of polyvinyl alcohol, polyurethane, ethyl cellulose, methyl cellulose, cellulose acetate, cellulose acetate butyrate, polyvinyl chloride, polycarbonate, ethylene-vinyl acetate, polymethyl methacrylate or polypropylene; the micronized water-soluble particles are selected from one or more of sodium chloride, sodium bicarbonate or mannitol; and the other additives comprise dibutyl phthalate, triethyl citrate, polyethylene glycol (PEG), talc, stearic acid, magnesium stearate or colloidal silica.

10. A method for preparing the film-coated tablet according to claim 1, wherein tablet core materials are mixed, tableted, and then coated to form a semipermeable film.

11. The film-coated tablet according to claim 4, wherein the drug is a drug with low solubility in acid; and the penetration enhancer is selected from one or more of potassium chloride, potassium sulfate, potassium hydrogen phosphate, sodium hydrogen phosphate, sodium chloride, citric acid or tartaric acid.

12. The film-coated tablet according to claim 4, wherein the gas generating agent is sodium bicarbonate, and the water-soluble swellable polymer is selected from hydroxypropyl methylcellulose, carbomer or polyethylene oxide.
